(19) United States
(12) Patent Application Publication
(10) Pub. No.: US 2020/0405402 A1
(43) Pub. Date: Dec. 31, 2020

(54) TOUCHLESS BUTTON CONTROL OF SURGICAL INSTRUMENT USING AUGMENTED REALITY

(71) Applicant: Covidien LP, Mansfield, MA (US)

(72) Inventors: Jordan A. Whisler, Brookline, MA (US); Paul F. Prokop, Woburn, MA (US)

(21) Appl. No.: 16/912,962

(22) Filed: Jun. 26, 2020

Related U.S. Application Data
(60) Provisional application No. 62/867,336, filed on Jun. 27, 2019.

(51) Int. Cl. A61B 34/00; A61B 17/32

(52) U.S. Cl. CPC A61B 34/25; A61B 2090/502; A61B 17/320016

(57) ABSTRACT

A surgical system that includes a surgical instrument, a display device, and a controller. The surgical instrument is configured for insertion at least partially into an internal body cavity of a patient, the surgical instrument having an adjustable setting. The controller is in communication with the surgical instrument and the display device. The controller includes a memory storing the adjustable setting of the surgical instrument and a processor. The processor reads from memory the adjustable setting of the surgical instrument and generates a virtual button corresponding to the adjustable setting of the surgical instrument. The generated virtual button is displayed and selectable by a user to implement a corresponding setting on the surgical instrument.

### CROSS-REFERENCE TO RELATED APPLICATIONS

This application claims the benefit of U.S. Provisional Application No. 62/867,336, filed Jun. 27, 2019, the entire contents of each of which are incorporated by reference herein.

### FIELD

The present technology is generally related to a surgical system, and more particularly to a surgical system with touchless button control using augmented reality.

### BACKGROUND

Many surgical instruments employ adjustable settings that the surgeon may dynamically change during a procedure. Typically, mechanical buttons and/or footswitches are utilized to adjust the adjustable settings. Additional buttons, actuators, etc., may be found on a console associated with the surgical instrument for adjusting other adjustable settings.

### SUMMARY

The techniques of this disclosure generally relate to a surgical system with touchless button control using augmented reality.

In aspects, the present disclosure provides a surgical system that includes a surgical instrument configured for insertion at least partially into an internal body cavity of a patient, a display device and a controller in communication with the surgical instrument and the display device. The controller includes a memory storing the adjustable setting of the surgical instrument, and a processor. The processor is configured to read from memory the adjustable setting of the surgical instrument, generate a virtual button corresponding to the adjustable setting of the surgical instrument, and display the virtual button on the display device. The displayed virtual button is selectable by a user to implement a corresponding setting on the surgical instrument.

In aspects, in displaying the virtual button, the display device may project the virtual button into the field of view.

In aspects, the display device may be a headset.

In aspects, in displaying the virtual button, the display device may project the virtual button onto at least one of: the surgical instrument, another surgical instrument, a monitor, or an available free space within the field of view.

In aspects, the surgical system may further includes an image capture device configured to capture movement to determine whether the virtual button has been selected.

In aspects, the image capture device may capture movement of a hand or a finger to an area where the virtual button may be projected to determine whether the virtual button has been selected.

In aspects, the image capture device may capture movement of eyes to an area where the virtual button may be projected to determine whether the virtual button has been selected.

In aspects, the surgical instrument may be a tissue resecting instrument, wherein the tissue resecting instrument may be activated according to the selected virtual button.

In aspects, selecting the virtual button may further cause the processor to generate a virtual adjustment button for the selected virtual button to adjust the selected adjustable setting. The processor may display the virtual adjustment button on the display device selectable by the user to adjust the selected adjustable setting to a desired state and set the surgical instrument to the desired state.

In aspects, the adjustable setting may include fluid pressure, blade movement, or blade speed.

In aspects, the processor may be further configured to display status information indicating the corresponding setting of the surgical instrument.

In another aspect, the disclosure provides a surgical instrument coupled to an image capture device configured to capture movement. In aspects of the disclosure the surgical instrument includes a controller in communication with a display device. The controller includes a memory storing an adjustable settings of the surgical instrument, and a processor. The processor is configured to read from memory the adjustable settings of the surgical instrument, generate a virtual button corresponding to the surgical instrument, and display the virtual button on the display device. The displayed virtual button is selectable by a user to implement a corresponding setting on the surgical instrument. The processor captures movement and generates a virtual adjustment button for the selected virtual button to adjust the selected adjustable setting. The processor displays the virtual adjustment button on the display device selectable by the user to adjust the selected adjustable setting to a desired state and set the surgical instrument to the desired state.

In aspects, the display device may be a headset.

In aspects, in displaying the virtual button or the virtual adjustment button, the display device may project the virtual button or the virtual adjustment button onto at least one of: the surgical instrument, another surgical instrument, a monitor, or an available free space within the field of view.

In aspects, the image capture device may capture movement of a hand, a finger, or eyes to an area where the virtual button or the virtual adjustment button may be projected to determine whether the virtual button or the virtual adjustment button has been selected.

In aspects, the surgical instrument may be a tissue resecting instrument, wherein the tissue resecting instrument may be activated according to the selected virtual button or virtual adjustment button to activate the fluid pressure, blade movement, or blade speed settings.

In aspects, the processor may further configured to display status information indicating the corresponding setting of the surgical instrument.

In another aspect, the disclosure provides a surgical system. In aspect of the disclosure the surgical system includes a tissue resecting instrument having an adjustable setting, an image capture device configured to capture movement, and a controller in communication with a display device. The controller includes a memory storing the adjustable setting of the tissue resecting instrument, and a processor. The processor is configured to read from memory the adjustable setting of the tissue resecting instrument and generate a virtual button corresponding to the adjustable setting of the resecting instrument. The virtual button is displayed on the display device selectable by a user to implement a corresponding setting on the tissue resecting instrument. The displayed virtual button is projected onto at least one of: the surgical instrument, another surgical instrument, a monitor, or an available free space within the field of view. The image capture device captures movement of eyes, hands, or fingers to an area where the virtual button is projected and the processor generates the virtual adjustment button for the selected adjustable setting configured to adjust the selected adjustable setting. The virtual adjustment button is displayed on the display device and selectable by the user to adjust the selected adjustable setting to a desired state. The displayed virtual adjustment button is projected onto at least one of: the surgical instrument, another surgical instrument, a monitor, or an available free space within the field of view. The image capture device captures movement of a hand, a finger, or eyes to an area where the virtual button or the virtual adjustment button is projected to determine whether the virtual button or the virtual adjustment button has been selected and sets the tissue resecting instrument to the desired state.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a block diagram of a surgical system incorporating augmented reality;

FIG. 2 is an exemplary diagram of the surgical system of FIG. 1 in use including touchless button; and

FIG. 3 is a perspective view of a surgical instrument of the surgical system of FIG. 1 operably positioned within an endoscope including touchless buttons.

### DETAILED DESCRIPTION

Particular embodiments of the disclosure are described hereinbelow with reference to the accompanying drawings. In the following description, well-known functions or constructions are not described in detail to avoid obscuring the present disclosure in unnecessary detail.

The following aspects of a surgical system, and in particular, a surgical system with touchless button control using augmented reality, incorporate features to enable access to several adjustable settings and information, e.g., of a surgical instrument in use, while maintaining surgeon interaction with the several adjustable settings without contact or requiring the surgeon to look away from surgical field.

The surgical systems incorporating augmented reality of the disclosure detailed below may be incorporated into different types of surgical systems, instruments, assemblies, or configurations. The particular illustrations and embodiments disclosed herein are merely exemplary and do not limit the scope or applicability of the disclosed technology.

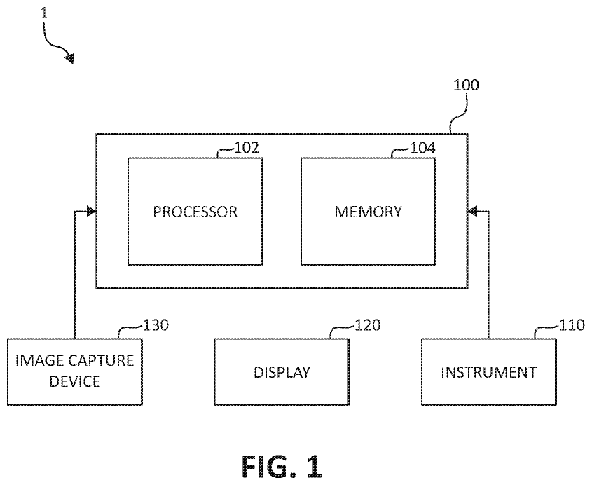

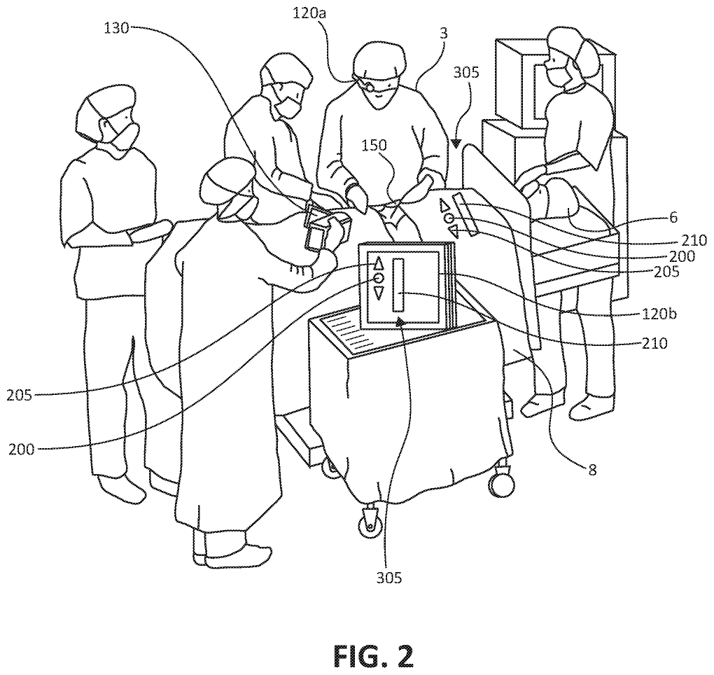

FIG. 1 illustrates a block diagram of a surgical system 1 and FIG. 2 illustrates an exemplary use of the surgical system 1. The surgical system 1 includes a controller 100 that has a processor 102 and a memory 104. The surgical system 1 also includes one or more surgical instruments 110, a display device 120, and an image capture device 130 operably coupled to the controller 100.

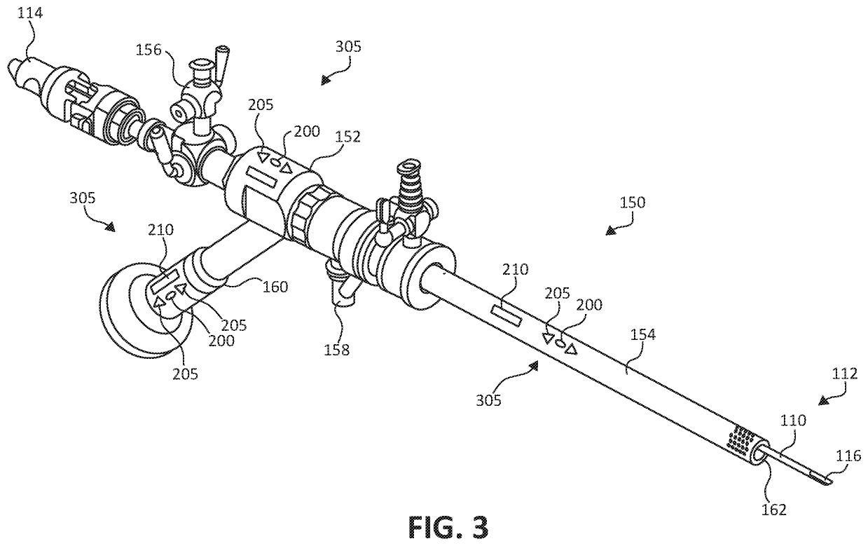

FIG. 3 illustrates an exemplary embodiment of the surgical instrument 110 of the surgical system 1 operably positioned within another surgical instrument, e.g., an endoscope 150. The surgical instrument 110 may be a tissue resecting instrument configured for insertion through endoscope 150 into an organ (e.g., a uterus, a prostate, a bladder, etc.) of a patient for use in a tissue resecting procedure in the organ. With respect to typical surgical instruments, the buttons for operating the surgical instruments are mechanical and out of reach from the user 3 (FIG. 2), such as, for example a footswitch or buttons located on a console coupled to the tissue resecting device. Mechanical buttons may be placed closer to the user 3 (FIG. 2) on the surgical instrument or other adjacent instruments; however, the presence of these buttons may make it difficult to seal the instruments properly for sterilization and do not allow for customization.

As noted above, surgical instrument 110 is shown disposed within an endoscope 150. Endoscope 150 includes a housing 152, an elongated sheath 154 extending distally from housing 152, one or more valves 156 for fluid input and/or output, a light post 158 extending transversely from housing 152, a visualization arm 160 obliquely angled relative to housing 152 and extending therefrom, and a visualization device 162 coupled to visualization arm 160 and extending through elongated sheath 154 to a distal end portion thereof.

The surgical instrument 110 includes an end effector assembly 112 and a proximal hub assembly 114. The proximal hub assembly 114 is configured to connect to a handpiece (not shown) which, in turn, is coupled to a control unit (not shown) via a cable to provide power and control functionality to surgical instrument 110, although surgical instrument 110 may alternatively or additionally include controls associated with the handpiece and/or a power source, e.g., battery, disposed within handpiece. The handpiece and/or control unit may further include a resector drive, e.g., a motor, configured to cause movement of a cutting blade 116 at the end effector assembly 112, to cut tissue from the organ. The surgical instrument 110 is further adapted to connect to a fluid management system (not shown) via outflow tubing for applying suction to remove fluid, tissue, and debris from a surgical site via the surgical instrument 110. The fluid management system may additionally or alternatively be coupled to the endoscope 150 to facilitate fluid inflow and/or outflow from the surgical site. The control unit and fluid management system may be integral with one another, coupled to one another, or separate from one another.

Turning back to FIG. 2, in conjunction with FIG. 1, illustrated is the surgical system 1 in use including one or more touchless buttons 305. In displaying a touchless button 305, the controller 100 causes the processor 102 to read from memory 104 an adjustable setting and/or status information relating to the surgical instrument 110, endoscope 150 (FIG. 3), the control unit, and/or the fluid management system. The processor 102 then generates one or more virtual operational buttons 200, e.g., an on/off button, and/or one or more virtual adjustment buttons 205, e.g., increase and decrease buttons, based on the one or more settings, collectively or individually, of the surgical instrument 110, endoscope 150 (FIG. 3), control unit, and/or fluid management system, such as, for example fluid pressure, blade movement, and blade speed. The processor 102 additionally or alternatively generates one or more virtual status indicators 210, e.g., numerical displays, charts, graphs, and/or icons, etc., based on the status information, collectively or individually, of the surgical instrument 110, endoscope 150 (FIG. 3), control unit, and/or fluid management system, such as, for example, a current fluid pressure, current blade movement profile, and current blade speed.

The display device 120, in communication with the processor 102 of the controller 100, is configured to project the virtual button 200, 205 and/or the status indicator 210 corresponding to the adjustable setting and the status information, respectively, of the surgical instrument 110, endoscope 150 (FIG. 3), control unit, and/or fluid management system. The display device 120 may be a pair of glasses that projects the image onto one of the lenses, such as, for example GOOGLE GLASS® (provided by Google®) both lenses, or on a facial shield, a headset 120a, the surgical instrument 110, the endoscope 150, a surgical drape 8, and/or a monitor 120b. With additional reference to FIG. 3, the display device 120 (FIG. 1) may project the touchless button 305, e.g., virtual button 200, 205 and/or virtual status indicator 210, onto one or more portions of at least one of the surgical instrument 110 within the field of view of the user 3, another surgical instrument 110, e.g., the endoscope 150, within the field of view of the user 3, the monitor 120b within the field of view of the user 3, or any available free space within the field of view of the user 3, such as, for example on a surgical drape 8 on the patient 6. The monitor 120b may, additionally or alternatively, overlay the touchless button 305 on an image of the patient obtained by the image capture device 130. Once the touchless button 305 is projected, the user 3 selects the touchless button 305 corresponding to the desired adjustable setting, e.g., to activate the control unit and/or the fluid management system and/or to adjust a setting thereof to set the surgical instrument 110 at a desired state. In various embodiments, instead of being projected, the touchless button 305 may be overlaid onto one or more portions of at least one of the surgical instrument 110, another surgical instrument 110, the monitor, or any available free space within the field of view of the user.

The selection of the touchless button 305 is monitored by the image capture device 130. The image capture device 130 captures movement of the user 3 during a surgical procedure. In capturing the movement of the user 3 during the surgical procedure, the image capture device 130 captures movement of a hand, a finger or eyes to an area in free space where the touchless button 305 is projected. In embodiments, once a touchless button 305 is selected, e.g., a virtual operational button 200, the processor 102 generates or reconfigures the touchless button 305, e.g., generating or more virtual adjustment buttons 205 and/or one or more virtual status indicators 210, corresponding to the selected virtual operational button 200. For example, where a virtual operation button 200 to activate the blade 116 of the surgical instrument 110 is selected, virtual adjustment buttons 205 to adjust the speed of the blade 116 of the surgical instrument 110 and/or virtual status indicators 210 indicating the current speed of the blade 116 may be generated.

The processor 102 in communication with the display device 120, displays the virtual adjustment buttons 200, 205 and/or virtual status indicators 210 as touchless button 305 on the display device 120. The display device 120 in communications with the controller 100 projects the corresponding virtual adjustment button(s) 205 and the virtual status indicator(s) 210. The virtual adjustment button(s) 205 and the virtual status indicator(s) 210 may be projected next to the virtual operational button 200 or onto at least one of the surgical instrument 110 within the field of view of the user 3, another surgical instrument 110 within the field of view of the user 3, the monitor 120b within the field of view of the user 3, or any available free space within the field of view of the user 3.

Alternatively, the controller 100 may read from memory 104 the adjustable setting of the surgical instrument 110 and the processor 102 may generate the virtual operational button 200, the virtual adjustment button 205, and the virtual status indicator 210 without prior selection from the user 3. The processor 102 in communication with the display device 120, in such embodiments, displays the virtual operational button 200, the virtual adjustment button 205, and the virtual status indicator 210 similarly as detailed above.

Upon selection of the virtual operational button 200 and/or the virtual adjustment button 205, the processor 102 transmits a signal to the surgical instrument 110, the endoscope 150, control unit, and/or the fluid management system to set the surgical instrument 110 to the desired state. The surgical instrument 110 is activated according to the desired state received by the processor 102 based on the selection of the virtual operational button 200 and/or the virtual adjustment button 205. Therefore, for example, the user 3 in a tissue resecting procedure may utilize the virtual operational button 200 and the virtual adjustment button 205 of the touchless button 305 in lieu of mechanical buttons resulting in ease of use, customizable use (wherein the user 3 can define the desired positions of the touchless buttons 305 or select from a plurality of layouts of touchless buttons 305), and efficient and proper sealing of the surgical instrument 110 for steam sterilization. Additionally, the user 3 can have access to the virtual operational button 200, the virtual adjustment button 205, and can view status information via the virtual status indicator 210 without looking away from the field of view during the surgical procedure.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A surgical system, comprising:
a surgical instrument configured for insertion at least partially into an internal body cavity of a patient, the surgical instrument having an adjustable setting;
a display device;
a controller in communication with the surgical instrument and the display device, the controller including:
a memory storing the adjustable setting of the surgical instrument; and
a processor configured to:
read from memory the adjustable setting of the surgical instrument;
generate a virtual button corresponding to the adjustable setting of the surgical instrument; and
display the virtual button on the display device, wherein the displayed virtual button is selectable by a user to implement a corresponding setting on the surgical instrument.

2. The surgical system of claim 1, wherein in displaying the virtual button, the display device projects the virtual button into the field of view.

3. The surgical system of claim 2, wherein the display device is a headset.

4. The surgical system of claim 1, wherein in displaying the virtual button, the display device projects the virtual button onto at least one of: the surgical instrument, another surgical instrument, a monitor, or an available free space within the field of view.

5. The surgical system of claim 1, further comprising an image capture device configured to capture movement to determine whether the virtual button has been selected.

6. The surgical system of claim 5, wherein the image capture device captures movement of a hand or a finger to an area where the virtual button is projected to determine whether the virtual button has been selected.

7. The surgical system of claim 5, wherein the image capture device captures movement of eyes to an area where the virtual button is projected to determine whether the virtual button has been selected.

8. The surgical system of claim 1, wherein the surgical instrument is a tissue resecting instrument, wherein the tissue resecting instrument is activated according to the selected virtual button.

9. The surgical system of claim 1, wherein selecting the virtual button further causes the processor to:
generate a virtual adjustment button for the selected virtual button, wherein the virtual adjustment button is configured to adjust the selected adjustable setting; and
display the virtual adjustment button on the display device, wherein the virtual adjustment button is selectable by the user to adjust the selected adjustable setting to a desired state;
set the surgical instrument to the desired state.

10. The surgical system of claim 1, wherein the adjustable setting includes fluid pressure, blade movement, or blade speed.

11. The surgical system of claim 1, wherein the processor is further configured to display status information indicating the corresponding setting of the surgical instrument.

12. A surgical instrument coupled to an image capture device configured to capture movement, the surgical instrument comprising:
a controller in communication with a display device, the controller including:
a memory storing an adjustable settings of the surgical instrument; and
a processor configured to:
read from memory the adjustable settings of the surgical instrument;
generate a virtual button corresponding to the surgical instrument;
display the virtual button on the display device, wherein the displayed virtual button is selectable by a user to implement a corresponding setting on the surgical instrument;
capture movement and generate a virtual adjustment button for the selected virtual button, wherein the virtual adjustment button is configured to adjust the selected adjustable setting;
display the virtual adjustment button on the display device, wherein the virtual adjustment button is selectable by the user to adjust the selected adjustable setting to a desired state; and
set the surgical instrument to the desired state.

13. The surgical instrument of claim 12, wherein the display device is a headset.

14. The surgical instrument of claim 12, wherein in displaying the virtual button or the virtual adjustment button, the display device projects the virtual button or the virtual adjustment button onto at least one of: the surgical instrument, another surgical instrument, a monitor, or an available free space within the field of view.

15. The surgical instrument of claim 12, wherein the image capture device captures movement of a hand, a finger, or eyes to an area where the virtual button or the virtual adjustment button is projected to determine whether the virtual button or the virtual adjustment button has been selected.

16. The surgical instrument of claim 12, wherein the surgical instrument is a tissue resecting instrument, wherein the tissue resecting instrument is activated according to the selected virtual button or virtual adjustment button to activate the fluid pressure, blade movement, or blade speed settings.

17. The surgical instrument of claim 12, wherein the processor is further configured to display status information indicating the corresponding setting of the surgical instrument.

18. A surgical system comprising:
a tissue resecting instrument having an adjustable setting;
an image capture device configured to capture movement; and
a controller in communication with the tissue resecting instrument and a display device, the controller including:
a memory storing the adjustable setting of the tissue resecting instrument; and
a processor configured to:
read from memory the adjustable setting of the tissue resecting instrument;
generate a virtual button corresponding to the adjustable setting of the resecting instrument;
display the virtual button on the display device, wherein the displayed virtual button is selectable by a user to implement a corresponding setting on the tissue resecting instrument;
project the virtual button onto at least one of: the surgical instrument, another surgical instrument, a monitor, or an available free space within the field of view;
capture movement of eyes, hands or fingers to an area where the virtual button is projected and generate virtual adjustment button for the selected adjustable setting, wherein the virtual adjustment button is configured to adjust the selected adjustable setting;
display the virtual adjustment button of the selected adjustable setting on the display device, wherein the virtual adjustment button is selectable by the user to adjust the selected adjustable setting to a desired state;
project the virtual adjustment button of the selected adjustable setting onto at least one of: the surgical instrument, another surgical instrument, a monitor, or an available free space within the field of view; and
capture movement of eyes, hands or fingers to an area where the virtual adjustment button is projected and set the tissue resecting instrument to the desired state.
